(19) United States
(12) Patent Application Publication
(10) Pub. No.: US 2013/0171086 A1
(43) Pub. Date: Jul. 4, 2013

(54) ANTIPERSPIRANT COMPOSITIONS AND METHODS FOR PREPARING ANTIPERSPIRANT COMPOSITIONS

(71) Applicants: Catherine Schmit, Glendale, AZ (US); Debra Butterworth, Scottsdale, AZ (US)

(72) Inventors: Catherine Schmit, Glendale, AZ (US); Debra Butterworth, Scottsdale, AZ (US)

(73) Assignee: THE DIAL CORPORATION, Scottsdale, AZ (US)

(21) Appl. No.: 13/340,339

(22) Filed: Dec. 29, 2011

(51) Int. Cl. A61K 8/44

(52) U.S. Cl. 424/66; 424/65

(57) ABSTRACT

Antiperspirant compositions with improved efficacy for shaving and methods for preparing the same are provided. In an exemplary embodiment, an antiperspirant composition includes an active antiperspirant compound, a hair minimizing compound, and a skin lubricating compound. In another exemplary embodiment, a method for preparing an antiperspirant composition includes combining an active antiperspirant compound, a suspending agent, and a carrier at a first temperature to form a mixture and cooling the mixture to form a cooled mixture having a second temperature that is lower than the first temperature. The exemplary method further includes combining a hair minimizing compound and a skin lubricating compound to the mixture and pouring the mixture into a mold at a third temperature that is lower than the second temperature.

### TECHNICAL FIELD

The present disclosure generally relates to antiperspirant compositions and methods for preparing antiperspirant compositions, and more particularly relates to antiperspirant compositions with improved antiperspirant efficacy due to hair shaft minimization and lubricated shaving and methods for preparing the same.

### BACKGROUND

Antiperspirants are popular personal care products used to prevent or eliminate perspiration and body odor caused by perspiration. Antiperspirant products, including for example sticks, emulsions, and roll-on antiperspirants are desired by a large majority of the population because of the presence of active antiperspirant compounds that minimize or prevent the secretion of perspiration by blocking or plugging ducts of sweat-secreting glands, such as those located at the underarms. Antiperspirants typically comprise an active antiperspirant compound in a carrier that permits the antiperspirant product to be applied to the skin by swiping or rubbing the stick across the skin, typically of the underarm. Upon application, the carrier coats the skin or evaporates, releasing the active antiperspirant compound from the antiperspirant product upon exposure to moisture to form plugs in the sweat ducts.

Active antiperspirant compounds reduce underarm wetness and odor by migrating into openings of the sweat gland ducts and reacting with proteins therein to form antiperspirant plugs, which mechanically prevent sweat from escaping the ducts. Two types of sweat glands are present in the underarm region. The first type of sweat gland, apocrine sweat glands, terminate and secrete at the top of hair follicles. As such, active antiperspirant compounds should migrate into the hair follicle to access the apocrine sweat gland duct and block secretion. However, underarm hair can partially block the duct opening, making it more difficult for the active antiperspirant compound to enter and migrate into the duct. The second type of sweat gland, eccrine sweat glands, open directly onto the skin. Eccrine sweat is responsible for the largest volume of sweat that causes underarm wetness. As with apocrine glands, active antiperspirant compounds migrate into the eccrine gland openings and form plugs, which reduce underarm wetness.

However, if the antiperspirant plugs form too near the skin surface, the plugs may be scraped off during underarm shaving. The coarser the user's hair, the more aggressive of shave that is required, and consequently the more antiperspirant plugs that may be removed. Furthermore, aggressive shaving can abrade the epidermis and remove additional antiperspirant plugs.

Accordingly, it is desirable to provide antiperspirant compositions that form plugs deeper within the glands to reduce the incidence of plug removal during underarm abrasion or shaving. Additionally, it is desirable to provide antiperspirant compositions that reduce the coarseness of hair such that less aggressive shaving is required, thereby reducing the likelihood that antiperspirant plugs will be removed during shaving. In addition, it is desirable to provide antiperspirant compositions that reduce skin abrasion during shaving, thereby reducing the likelihood that antiperspirant plugs will be removed. Further, it is desirable to provide methods for preparing these antiperspirant compositions. Furthermore, other desirable features and characteristics of the present disclosure will become apparent from the subsequent detailed description the appended claims, taken in conjunction with the accompanying drawings and background.

### BRIEF SUMMARY

Antiperspirant compositions with improved efficacy due to hair shaft minimization and gentler shaving and methods for preparing the same are provided. In an exemplary embodiment, an antiperspirant composition includes an active antiperspirant compound, a hair minimizing compound, and a skin lubricating compound. The hair minimizing compound may include a compound including urea and salicylic acid. The skin lubricating compound may include an emollient. The active antiperspirant compound may be an aluminum zirconium trichlorohydrex glycine complex.

In another exemplary embodiment, a method for preparing an antiperspirant composition includes combining an active antiperspirant compound, a suspending agent, and a carrier at a first temperature to form a mixture and cooling the mixture to form a cooled mixture having a second temperature that is lower than the first temperature. The exemplary method further includes combining a hair minimizing compound and a skin lubricating compound to the mixture and pouring the mixture into a mold at a third temperature that is lower than the second temperature.

In yet another exemplary embodiment, an antiperspirant composition includes an active antiperspirant compound including an aluminum zirconium trichlorohydrex glycine complex, a hair minimizing compound including urea and salicylic acid, and a skin lubricating compound including silk powder and aloe vera.

### DETAILED DESCRIPTION

The following detailed description is merely exemplary in nature and is not intended to limit the disclosed embodiments or the application and uses of the disclosed embodiments. Furthermore, there is no intention to be bound by any theory presented in the preceding background or the following detailed description.

The various embodiments contemplated herein relate to antiperspirant compositions and methods for preparing antiperspirant compositions. The various embodiments of the compositions exhibit improved antiperspirant and deodorant properties during and after shaving. That is, when applied to the skin of a user, for example the underarm area of the user, the antiperspirant compositions retains desirable antiperspirant and deodorant properties after the underarm has been shaved.

The inventors have found that antiperspirant compositions that exhibit improved efficacy for shaving can be prepared by utilizing the synergistic effects of two compounds when added to the antiperspirant composition. The two additional compounds to be added to the antiperspirant composition in accordance with the present disclosure include a hair minimizing compound and a skin lubricating compound. It has been unexpectedly found that improved sweat plug formation is achieved by adding a hair minimizing compound to the antiperspirant formulation, minimizing underarm hair coarseness, thus increasing the access of an active antiperspirant compound to the hair follicle and the apocrine sweat gland duct. Synergistically, improved antiperspirant plug maintenance is also achieved by minimizing coarseness of hair, making it easier to shave the underarm by requiring a less aggressive shave. In addition, it has been discovered that the inclusion of a skin lubricating compound in the antiperspirant formulation provides lubrication or “slip” for shaving, reducing friction on the skin surface and thereby increasing the ease of shaving and reducing the abrasion that occurs during shaving.

The combined synergistic effect of improved antiperspirant plug formation due to finer hair, less aggressive shaving also due to finer hair, and less aggressive shaving and less abrasion due to improved slip across the skin, has thus been discovered to result from the addition of a hair minimizing compound and a lubricating compound to an antiperspirant composition. Particular formulations thereof, and methods for making the same, are described hereinafter.

In one embodiment, an antiperspirant composition in accordance with the present disclosure includes a water-soluble active antiperspirant compound. Active antiperspirant compounds contain at least one active ingredient, for example metal salts, that, as noted above, are thought to reduce perspiration by diffusing through the sweat ducts of apocrine glands and eccrine glands and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used in the antiperspirant product include astringent metallic salts, for example inorganic and organic salts of aluminum, zirconium, and zinc, including tetra- and octa-salts, as well as mixtures thereof. Exemplary compounds include aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Exemplary zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In one embodiment, the active antiperspirant compound is aluminum zirconium pentachlorohydrex glycine complex or aluminum zirconium trichlorohydrex glycine complex. In a further embodiment, the antiperspirant product comprises an active antiperspirant compound in an amount of about 8 to about 30 wt. % (USP). As used herein, weight percent (USP) or wt. % (USP) of an antiperspirant salt is calculated as anhydrous weight percent in accordance with the U.S.P. method, as is well known in the art. This calculation excludes any bound water and glycine. In yet another embodiment, the antiperspirant composition includes about 20-25 wt. % aluminum zirconium pentachlorohydrex glycine complex or aluminum zirconium trichlorohydrex glycine complex.

In one embodiment, an antiperspirant composition in accordance with the present disclosure further includes at least one structurant and/or gellant (hereinafter referred collectively as “structurant”) that facilitates the solid consistency of the antiperspirant stick product. Naturally-occurring or synthetic waxy materials or combinations thereof can be used as such structurants. Suitable structurants, including waxes and gellants, are often selected from fatty alcohols often containing from 12 to 30 carbons, such as stearyl alcohol, behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. In an exemplary embodiment, a mixture of wax structurants may be employed. Suitable mixtures of structurants can reduce the visibility of active antiperspirant compounds deposited on the skin and result in either a soft solid or a firm solid. In an exemplary embodiment, the surfactant(s) comprise about 10 to about 35 wt. % of the total antiperspirant composition. In a preferred embodiment, the antiperspirant composition comprises a mixture of stearyl alcohol and hydrogenated castor oil. In another exemplary embodiment, the antiperspirant composition comprises about 12 to about 25 wt. % stearyl alcohol and about 1.5 to about 7 wt. % hydrogenated castor oil. In yet another exemplary embodiment, the antiperspirant composition comprises about 15-22 wt. % stearyl alcohol and about 2.8 wt. % hydrogenated castor oil.

In one embodiment, an antiperspirant composition in accordance with the present disclosure further includes a high refractive index (R.I.) hydrophobic compound. As used herein, the term “high refractive index” means a refractive index of no less than about 1.4. The high R.I. hydrophobic compound also facilitates the minimization and/or prevention of a white residue on the skin by masking the active antiperspirant salt that stays upon the skin upon evaporation of the carrier. Examples of high R.I. hydrophobic compounds for use in the antiperspirant products include PPG-14 butyl ether, C₁₂-C₁₅ alkyl benzoate, such as Finsolv TN® available from Innospec of the United Kingdom, and phenyl dimethicone. In a preferred embodiment, the antiperspirant composition comprises PPG-14 butyl ether and, in a more preferred embodiment, the antiperspirant composition comprises PPG-14 butyl ether in an amount of about 5 to about 15 wt. % of the total antiperspirant composition. In a most preferred embodiment, the antiperspirant product comprises about 9 to about 11 wt. % PPG-14 butyl ether.

In one exemplary embodiment, the antiperspirant product includes one or more suspending agents that facilitate suspension of the active antiperspirant compound in the antiperspirant product, thereby minimizing the amount of active antiperspirant compound that settles out of the antiperspirant product during manufacture. Suitable suspending agents include clays and silicas. Examples of suitable silicas include fumed silicas and silica derivatives, such as silica dimethyl silylate. Suitable clays include bentonites, hectorites and colloidal magnesium aluminum silicates. In one exemplary embodiment, the antiperspirant product includes about 0.2 to about 2.5 wt. % suspending agents. In another exemplary embodiment, the antiperspirant product comprises a mixture of silica and silica dimethyl silylate. In a further embodiment, the antiperspirant product comprises from about 0.1-0.5 wt. % silica and from about 0.1 to about 2 wt. % silica dimethyl silylate. In another yet another embodiment, the antiperspirant product does not use suspending agents, but comprises high melting point waxes to prevent settling of the active ingredients. Examples of suitable high melting point waxes include hydrogenated castor oils and polyethylenes having various melting points above 65° C.

In addition to the compounds identified above, the antiperspirant product may include additives, such as those used in conventional antiperspirants. These additives include, but are not limited to, fragrances, including encapsulated fragrances, dyes, pigments, preservatives, antioxidants, moisturizers, and the like. These ingredients can be included in the antiperspirant product in an amount of 0 to about 20 wt. %. In an exemplary embodiment, the antiperspirant product includes myristyl myristate, which provides a conditioning effect to the skin.

The antiperspirant product, in one embodiment, further includes a hydrophobic carrier. Exemplary hydrophobic carriers include liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Exemplary siloxanes include cyclomethicones, which have from about 3 to about 6 silicon atoms, such as cyclotetramethicone, cyclopentamethicone, and cyclohexamethicone, and mixtures thereof. The carrier also may include, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

In one embodiment, the antiperspirant product further includes a hair minimizing compound. As set forth above, the hair minimizing compound serves to minimize the hair in, for example, the underarm area of the user, thereby allowing the active antiperspirant compound easier access to the sweat gland duct and further reducing the aggressiveness of shaving required. Exemplary hair minimizing compounds include, but are not limited to, urea, salycilic acid, and salix alba (willow) bark extract. Further hair minimizing compounds that may be employed with active antiperspirant compounds include, but are not limited to, myrica cerifera (bayberry) leaf extract, dihydromyricetin, cymnema sylvestre leaf extract, Jserenoa serrulata (saw palmetto) fruit extract, epilobium angustifolium (fireweed) extract, cucurbita pepo (pumpkin) seed extract, larrea divaricata (chaparral) extract, palmatine, subtilisin, hydrolyzed soy protein, nordihydroguaiaretic acid, acetyl glucosamine, and oleanolic acid. A particular hair minimizing compound known in the art is Pilinhib™, manufactured by Cognis Corporation, which includes propylene glycol, hydrolyzed soy protein, hypericum performatum extract, hamaelis virginiana extract, arnica montana flower extract, urea, willow bark extract, menthol, and salicylic acid. The hair minimizing compound may be added to the antiperspirant composition in an amount between about 0.1-20% by weight, about 0.5-10% by weight, or about 1-5% by weight of the total antiperspirant composition.

In one embodiment, the antiperspirant composition further includes a skin lubricating compound. As set forth above, the skin lubricating compound serves to reduce the aggressiveness and increase the ease with which the user shave, and further to reduce skin abrasion during shaving, thereby minimizing the number of antiperspirant plugs removed during shaving. Exemplary skin lubricating compounds include emollients, which serve to soften or smooth the user's skin prior to and after shaving. In an exemplary embodiment, skin lubricating compounds include, but are not limited to, silk powder or aloe vera. Exemplary skin lubricating compounds further include slip modifiers, which are compounds used to enhance the flow properties of other compounds but do not react chemically with the compounds to which they are added. Numerous slip modifying compounds are known in the art, and include quaternary ammonium compounds (including salts), synthetic polymers (including salts), glycosaminoglycans, fatty esters, hydrocarbons, i.e., oils and waxes, silicone polymers, siloxanes and silanes, soaps, and fatty esters. The skin lubricating compound may be added to the antiperspirant composition in an amount between about 1-20% by weight, about 1-10% by weight, or about 1-5% by weight of the total antiperspirant composition. An exemplary, non-limiting listing of skin lubricating compounds thereof is set forth in Table 1, below.

TABLE 1
Acrylamide/Ethalkonium Chloride Acrylate Copolymer
Acrylamide/Ethyltrimonium Chloride Acrylate
Acrylic Acid/VP Crosspolymer
Acrylonitrile/Methyl Methacrylate Copolymer
Adipic Acid/Neopentyl Glycol Crosspolymer
Algae Exopolysaccharides
Behenyl Olivate
Bis-Aminopropyl Dimethicone/IPDI Copolymer
Bis-Hydroxyethoxypropyl Dimethicone Beeswax Esters
Bis-Hydroxyethoxypropyl Dimethicone Isostearate
Bis-Vinyl Dimethicone/Dimethicone Copolymer
Bis-Vinyldimethicone/PEG-10 Dimethicone Crosspolymer
Bletilla Striata Root Powder
Boron Nitride
Brassica Alcohol
Butene/Propylene Copolymer
Butylethylpropanediol Dimer Dilinoleate
C4-24 Alkyl Dimethicone/Divinyldimethicone Crosspolymer
Cannabis Sativa Stem Powder
Cellulose
Cetrimonium Dimethicone PEG-7 Phthalate
Chalk
Coco/Sunfloweramidopropyl Betaine
C24-30 Olefin
C28-36 Olefin
Dibutyl Fumarate/Hexadecene/Tetradecene Copolymer
Dibutyl Maleate/Hexadecene/Tetradecene Copolymer
Diisoamyl Malate
Dipentaerythrityl Tetrabehenate/Polyhydroxystearate
Diphenyl Dimethicone Crosspolymer
Ditrimethylolpropane Tetra Palm Kernelate
Glyceryl Diethylsiloxanes
Glyceryl Dimethylsiloxanes
Glycol Dimethacrylate Crosspolymer
Gossypium Herbaceum (Cotton) Powder
Hexafluoropropylene/Tetrafluoroethylene Copolymer
Hibiscus Cannabinus Stem Powder
Hydrogenated Camellia Oleifera Seed Oil
Hydrogenated Evening Primrose Oil
Hydrogenated Grapeseed Oil
Hydrogenated Hazelnut Oil
Hydrogenated Hemp Seed Oil
Hydrogenated Ostrich Oil
Hydrogenated Sesame Seed Oil
Hydroxycyclohexyl Ethyltrisiloxane Sapucainhate
Kaolin
Lauryl Methacrylate/Sodium Methacrylate Crosspolymer
Lauryl Phenylisopropyl Methicone
Lauryl Trimethicone
Lithium/Potassium/Iron/Titanium Oxides
Lithium/Potassium/Titanium Oxides
Lithium Stearate
Magnesium Aluminum Silicate
Magnesium Cocoate
Magnesium Myristate
Magnesium Palmitate
Myristyl Trisiloxane
Nephrite Powder
Octyldodecyl Olivate
PEG-6 Dimethicone
PEG-4 Distearyl Ether
PEG-200 Hydrogenated Castor Oil/IPDI Copolymer
PEG/PPG-12/16 Dimethicone
PEG/PPG-12/18 Dimethicone
PEG/PPG-16/8 Dimethicone
PEG/PPG-20/23 Dimethicone
PEG/PPG-23/6 Dimethicone
Perfluorocyclohexylmethanol
Perfluoroheptane
Perfluoromethylcyclohexane
Perfluoromethyldecalin
Perfluorononyl Dimethicone
Perfluorononylethyl Dimethicone/Methicone Copolymer
Phaseolus Radiatus Seed Powder
Phenylisopropyl Dimethicone
Pisum Sativum (Pea) Starch
Polydiethylsiloxane
Polyester-11
Polyethyloxazoline
Polyglyceryl-3 Behenate
Polyglyceryl-6 Behenate
Polyperfluoroethoxymethoxy Difluoroethyl PEG
Polyperfluoroethoxymethoxy Difluoroethyl PEG Ether
Polyurethane-16
Polyvinylidene Difluoride
Potassium Behenoyl Hydrolyzed Rice Protein
PPG-3 Benzyl Ether Ethylhexanoate
PPG-3 Benzyl Ether Myristate
PTFE
Pueraria Lobata Starch
Raffinose Isostearate
Saccharomyces/Opal/Mother of Pearl/Turquoise
Shea Butter Glycerides
Silica Dimethyl Silylate
Silk Powder
Sodium/Aluminum Hydroxide/Oxalate/Sulfate
Sodium Stearyl Fumarate
Stearyl Trimethicone
Synthetic Sapphire
Talc
Titanium Nitride
Tri-PPG-3 Myristyl Ether Citrate
Undecylcrylene Dimethicone
Zinc Laurate
Zinc Myristate
Zinc Palmitate
Zinc Rosinate
Zinc Stearate

The antiperspirant composition, in an exemplary embodiment, is prepared by combining the suspending agents in the hydrophobic carrier. Any suitable form of mixing can be used to combine the ingredients, such as high shear mixing, stirring, agitation, blending, or any combination thereof. The active antiperspirant compound is added to the suspending agents and carrier to form a premix. Mixing continues until the premix is homogenous and fluid in consistency. The structurants and the high refractive index (R.I.) hydrophobic compound, if used, are added to a mixing vessel and heat not exceeding about 85° C. is applied to melt the ingredients. As the ingredients melt, agitation is slowly commenced. The mixture, if necessary, is further cooled to about 64-69° C. and, with continuous agitation, the premix is incrementally added to the mixture until the mixture is homogenous. Additional carrier is added to the mixture with agitation such that the mixture is maintained at a temperature of about 60° C. Additives, including the hair minimizing compound and the skin lubricating compound, and further including compounds such as fragrances, dyes, corn starch, etc., are added with mixing while maintaining the mixture at 60° C. The final mixture is cooled to about 53° C., poured into molds, and then allowed to cool to room temperature. As used herein, the term “allowed to cool” means exposing the mixture to room temperature for a time sufficient for the mixture to come to room temperature or exposing the mixture to a refrigerator or cooling room, fan, or other cooling mechanism that lowers the temperature of the mixture to room temperature. It will be appreciated that the sequence of addition and/or combination of the various components of the antiperspirant product is not necessarily critical, and various sequences for addition or combination of the components can be used.

The following is an exemplary embodiment of an antiperspirant composition contemplated herein, with each of the components set forth in weight percent of the antiperspirant product. The example is provided for illustration purposes only and is not meant to limit the various embodiments of the antiperspirant product in any way.

### EXAMPLE

| Ingredient | Wt. % |
|---|---|
| Cyclopentasiloxane (245 Fluid) | 25.22 |
| Stearyl Alcohol | 24.00 |
| Ultrex 9 Salt | 16.00 |
| Cyclopentasiloxane | 15.91 |
| PPG-14 butyl ether | 9.80 |
| Hydrogenated castor oil | 2.84 |
| Myristyl Myristate | 1.92 |
| Aerosil R 972 V ™ | 1.01 |
| Performathox 450 ™ | 1.00 |
| Pilinhib (Cognis) ™ | 1.00 |
| QST Q-16086/1 Aloe | 0.85 |
| Silica (Aerosil 300 ™) | 0.25 |
| Silk Powder | 0.10 |
| Aloe Extract #104 | 0.10 |
| Total | 100.00 |

wherein Cyclopentasiloxane 245 Fluid ™ is available from Dow Corning of Midland Township, MI, Performathox 450 ™ is available from New Phase Technologies of Sugar Land, Aerosil R 972 V ™ and Aerosil 300 ™ are available from Evonik Industries AG of Essen, Germany, and Pilinhib ™ is available from Cognis Corporation of Cincinnati, OH.

A 1000 g sample was prepared in accordance with the above Example. The sample was prepared by adding 159.1 grams cyclopentasiloxane and 252.2 grams cyclopentasiloxane 245 Fluid (a cyclopentasiloxane composition manufactured by Dow Corning to have a slower evaporation rate) to a mixing vessel and commencing agitation. With agitation, 2.5 grams silica was incrementally added to the cyclopentasiloxane and agitation continued until the silica was wetted. Utilizing a high shear mixer, 160.0 grams Ultrex 9 was added to the suspending agents and carrier to form a premix. Mixing continued until the premix was homogenous and fluid in consistency. 240.0 grams stearyl alcohol, 28.4 grams hydrogenated castor oil, 98.0 grams PPG-14 butyl ether, 10.1 grams Aerosil R 972 V™, 10 grams Performathox™, and 1.92 grams myristyl myristate were added to a mixing vessel and heat not exceeding 85° C. was applied to melt the ingredients. As the ingredients melted, agitation was slowly commenced. Once the mixture was molten, it was cooled to 65-75° C. The mixture, was then further cooled to 64-69° C. and, with continuous agitation, the premix was incrementally added to the mixture until the mixture was homogenous. 1 gram each of silk powder and aloe extract, 8.5 grams QST Q-16086/1 Aloe, and 10 grams Pilinhib™ were was added with mixing while maintaining the mixture at 60° C. The final mixture was cooled to 53° C., poured into molds, and then allowed to cool to room temperature.

Accordingly, various embodiments of antiperspirant composition exhibiting increased efficacy during and after shaving that include a hair minimizing compound and a skin lubricating compound have been provided. The antiperspirant products exhibit improved antiperspirant plug formation due to finer hair, less aggressive shaving also due to finer hair, and less aggressive shaving and less abrasion due to improved slip across the skin.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the antiperspirant compounds in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

What is claimed is:

1. An antiperspirant composition comprising:
an active antiperspirant compound;
a hair minimizing compound; and
a skin lubricating compound.

2. The antiperspirant composition of claim 1, wherein the hair minimizing compound is a compound comprising urea and salicylic acid.

3. The antiperspirant composition of claim 1, wherein the skin lubricating compound is an emollient.

4. The antiperspirant composition of claim 3, wherein the emollient comprises silk powder and aloe vera.

5. The antiperspirant composition of claim 1, wherein the skin lubricating compound is a slip modifying compound.

6. The antiperspirant composition of claim 1, wherein the active antiperspirant compound is an aluminum zirconium trichlorohydrex glycine complex.

7. The antiperspirant composition of claim 1, wherein the active antiperspirant compound is aluminum zirconium pentachlorohydrex glycine complex.

8. The antiperspirant composition of claim 1, further comprising a hydrophobic carrier.

9. The antiperspirant composition of claim 8, wherein the hydrophobic carrier is a cyclopentasiloxane compound.

10. The antiperspirant composition of claim 1, further comprising a mixture of stearyl alcohol and hydrogenated castor oil.

11. The antiperspirant composition of claim 1, further comprising a high refractive index hydrophobic compound.

12. A method of manufacturing an antiperspirant composition, the method comprising:
combining an active antiperspirant compound, a suspending agent, and a carrier at a first temperature to form a mixture;
cooling the mixture to form a cooled mixture having a second temperature that is lower than the first temperature;
combining a hair minimizing compound and a skin lubricating compound with the mixture; and
pouring the mixture into a mold at a third temperature that is lower than the second temperature.

13. The method of claim 12, wherein combining a hair minimizing compound with the mixture comprises combining hydrolyzed soy protein and salicylic acid with the mixture.

14. The method of claim 12, wherein combining a skin lubricating compound with the mixture comprises combining an emollient with the mixture.

15. The method of claim 14, wherein combining an emollient with the mixture comprises combining silk powder and aloe vera with the mixture.

16. The method of claim 12, wherein combining a skin lubricating compound with the mixture comprises combining a slip modifying compound with the mixture.

17. The method of claim 12, wherein combining an active antiperspirant compound comprises combining an aluminum zirconium pentachlorohydrex glycine complex.

18. The method of claim 12, wherein combining a suspending agent comprises combining silica.

19. The method of claim 17, wherein combining a carrier comprises combining a cyclopentasiloxane compound.

20. An antiperspirant composition comprising:
an active antiperspirant compound comprising an aluminum zirconium trichlorohydrex glycine complex;
a hair minimizing compound comprising urea and salicylic acid; and
an skin lubricating compound comprising silk powder and aloe vera.
